(19) United States
(12) Patent Application Publication
(10) Pub. No.: US 2014/0051989 A1
(43) Pub. Date: Feb. 20, 2014

(54) DEVICES ADAPTED FOR ULTRASOUND LOCATION IN PATIENTS AND METHOD OF USE

(71) Applicants: Jane E. McGowan, Merion Station, PA (US); Peter A. Lewin, Wyndmoor, PA (US); Christopher Bawiec, Philadelphia, PA (US); Youhan Sunny, Philadelphia, PA (US)

(72) Inventors: Jane E. McGowan, Merion Station, PA (US); Peter A. Lewin, Wyndmoor, PA (US); Christopher Bawiec, Philadelphia, PA (US); Youhan Sunny, Philadelphia, PA (US)

(73) Assignee: DREXEL UNIVERSITY, Philadelphia, PA (US)

(21) Appl. No.: 14/008,967

(22) PCT Filed: Apr. 19, 2012
(86) PCT No.: PCT/US12/34185

§ 371 Date: Sep. 30, 2013

Related U.S. Application Data
(60) Provisional application No. 61/476,940, filed on Apr. 19, 2011.

(51) Int. Cl. A61B 5/06; A61M 25/00; A61N 5/10; A61M 39/00; A61B 8/00; A61M 16/04

(52) U.S. Cl. CPC A61B 5/061; A61B 8/00; A61M 16/04; A61N 5/1001; A61M 39/00; A61M 25/00
USPC 600/424

(57) ABSTRACT

The present invention relates to devices including a plurality of voids that enhance visualization of the devices in a patient using ultrasound imaging. The sizes of the voids can vary to accommodate ultrasound devices having different ultrasound wave frequencies. The present invention is also directed to a method for using ultrasound imaging technology to detect the location of devices comprising a plurality of voids in a patient. An ultrasound device, such as a wireless, portable ultrasound device, may be used to propagate ultrasound waves towards the patient where the device is inserted. An ultrasound imaging device may then be used to generate an image of the device or a portion thereof from which the location of the device in the patient can be determined.

### BACKGROUND OF THE INVENTION

1. Field of the Invention

The present invention relates generally to devices adapted for ultrasound imaging. More specifically, the present invention relates to devices and methods for using ultrasound imaging for verification and monitoring of the location of a device in a patient.

2. Description of the Related Technology

Many devices are inserted into the body of patients for facilitating diagnosis and treatment. The proper use and functioning of such devices frequently depends on accurate positioning of the device in the patient. Current methods for determining the position of devices in patients suffer from disadvantages such as insufficient resolution to accurately identify the device location, the use of harmful x-rays to obtain positioning information and the lack of simple, portable devices that can be employed for device location in a patient.

For example, the endotracheal tube (ETT) is routinely used in intensive care units, emergency rooms and other healthcare settings to restore and maintain an adequate air flow into the lungs of a patient. In a typical endotracheal intubation procedure, the distal end of the ETT is inserted into the trachea of a patient, generally at a location midway between the vocal folds and the carina. The proximal end is connected with a ventilation unit to supply air to the lungs.

Ensuring that the ETT is at the right location in the airway is critical for the success of the endotracheal intubation procedure. Adverse effects are serious if the ETT is at the a suboptimal location, ranging from filling the stomach with air and, therefore, not ventilating the lungs if the ETT is misplaced in the esophagus to ventilating only one lung if the ETT is placed too deeply into a bronchus. In instances where only one lung is ventilated, the ventilated lung may become overdistended, causing barotrauma to the ventilated lung and atelectasis in the contralateral lung

The commonly used technology for determining the position of the ETT in the airway of a patient after its placement is by X-ray imaging of the patient's neck and chest. Although X-ray imaging can accurately locate the ETT in airway of a patient, this methodology has a number of drawbacks. Namely, X-ray imaging does not enable real-time determination of the location of the ETT. Moreover, the negative impact on the patient is significant, including unnecessary ionizing radiation exposure, as well as unnecessary lifting and arranging of the patient for the X-ray imaging. The repeated exposures to ionizing radiation in the form of X-rays may be harmful to small babies, especially neonates who are still developing and thus at risk for possible long-term adverse effects

The development of an ultrasound method for localizing the position of an ETT within the airway will allow practitioners to check the position of the ETT without using X-rays. In many patients, but particularly in neonates, the position of the tube needs to be frequently checked as the ETT may shift without deliberate intervention and a slight shift, even of a few millimeters, can lead to compromised respiratory function, especially in neonates. An ultrasound based device and method would obviate the need for most X-ray imaging thereby reducing exposure to ionizing radiation which results from the current standard method.

The invention addresses the difficulty in locating and monitoring devices such as an ETT using ultrasound imaging systems. Although ultrasound techniques are noninvasive and can generate real time images of the device in the body, these images are generally poor in quality because of the presence of air within the immediate vicinity of the device to be located, including its tip, causing relatively poor echo amplitude strength. Poor quality ultrasound images, especially the low contrast between the device and surrounding tissue, make it challenging to correctly locate the device in the patient's body from conventional ultrasound images.

Several prior art references have offered partial solutions to enhance the quality of ultrasound images. U.S. Patent application no. 2006/0081255, for example, describes a system using a vibration mechanism coupled to the distal end of an ETT. By vibrating the ETT, it is possible to generate a slight phase shift in the frequency of the reflected ultrasound wave, which helps to improve the ultrasound imaging quality. This system, however, only permits identification of the ETT by virtue of its vibration, which can be difficult to discern and may cause unnecessary trauma to a patient. Vibration is especially problematic for use in neonates, because the tissue of a neonate is very fragile and vibration of the ETT may cause damage to the airway. In addition, vibration itself may cause shift of ETT to an undesirable location, since a shift of the ETT by a few millimeters may compromise respiratory function for neonates.

Another option is the use of a cuff affixed to a distal portion of the exterior tube wall of an ETT for enhanced imaging of the cuff [Raphael, David et. al., “Ultrasound confirmation of endotracheal tube placement,” Journal of Clinical Ultrasound, Vol. 15, issue 7, pp. 459-462 (December 2005)]. The cuff may be constructed as a saline filled balloon. Alternatively, the cuff may be configured as a spongy foam cuff containing air cells that are inflated after intubation to seal the trachea. Because the cuff is large, the longitudinal profile of the cuff is generally easier to identify than the distal end of the ETT when viewed by ultrasonic imaging. Furthermore, longitudinal movement of the ETT facilitates visualization of the spongy foam cuff The image quality of these cuffs, however, is limited and still requires interpretation by a trained eye to accurately identify the location of the ETT. Furthermore, it needs to be particularly emphasized that such cuffs are not suitable for use in neonates for fear of causing trauma to the airway.

The viability of using ultrasound imaging to identify the position of an ETT in neonates has also been evaluated. Slovis, T L, et. al. “Endotracheal tubes in neonates: sonographic positioning” Radiology, July vol. 160 no. 1 (July 1986), for example, relied on the motion of an ETT in the airway of a neonate caused by quickly moving the tube back and forth to provide a limited means for locating the ETT. In Lingle, Peter Allen, “Sonographic verification of endotracheal tube position in neonates: A modified technique” Journal of Clinical Ultrasound, vol. 16, no. 8, (October 1988), the manubrium of an intubated neonate was palpated and a standoff patch was positioned on the infant's neck and chest that functioned as a reference point for identifying the distal tip of an ETT. Both studies required movement of the ETT to enable location of the ETT and still produced poor ultrasound images that required an ultrasound specialist to interpret the ultrasound images. This method is also highly undesirable for use in neonates since vibration of the ETT may cause tissue damage to the relatively vulnerable tissue of such neonates. Also, the quality of the ultrasound image generated in this manner is not sufficient to enable healthcare providers lacking specialized training in ultrasound imaging to reliably use this technique to verify the location of the ETT.

The aforementioned ultrasound methods all generate relatively poor quality images of the ETT and surrounding tissue with little contrast between the ETT and the tissue. Using these methods, it is difficult to distinguish the ETT from the background, and thus trained personnel are required to interpret these images. Due to the poor quality of these images, however, even an ultrasound specialist may, in some cases, not be able to accurately discern the exact location of the ETT.

In another application, contrast agent microbubbles have been used to enhance the quality of ultrasound images in a patient's circulatory system. U.S. Pat. No. 6,086,540, for example, describes a method of injecting a gas contrast agent that forms microbubbles into a patient's blood stream for the purpose of analyzing cardiac circulation. These microbubbles reflect ultrasound waves that increase the contrast between the blood and the surrounding tissues. These references, however, do not suggest how to employ such microbubbles to visualize a device or using microbubbles to locate and monitor the position of a device within a patient.

Current standard techniques for determining the position of an ETT typically require multiple exposures to ionizing radiation in the form of X-rays. The radiation is especially harmful to neonates. The prior attempts to use ultrasound imaging techniques for locating the ETT are not sufficiently safe for use in neonates or sufficiently reliable for use by healthcare providers lacking specialized training in ultrasound imaging. Therefore there is a need to develop an improved noninvasive procedure which does not employ ionizing radiation for locating and/or monitoring the location of a device, such as an ETT as well as improved devices that can be more clearly visualized by a healthcare provider using ultrasound imaging.

### SUMMARY OF THE INVENTION

In a first aspect, the present invention relates to a device that includes a plurality of voids located in the tube wall in an amount sufficient for locating the device in a patient using ultrasound imaging.

In a second aspect, the present invention relates to a method of using ultrasound imaging to detect the location of a device having a plurality of voids located therein in patient's body, including the steps of: propagating an ultrasound wave through a location in the patient where the device is inserted; receiving the reflected ultrasound wave; generating an ultrasound image and locating the device using the ultrasound image.

### BRIEF DESCRIPTION OF THE DRAWINGS

FIG. 1 is a diagram of an endotracheal tube inserted into the airway of a patient. The proximal end of the tube is connected to a ventilation unit.

FIG. 2 is a longitudinal cross-sectional view of an endotracheal tube in accordance with a first embodiment of the present invention looking at the tube in the same direction as in FIG. 1.

FIG. 3 is a longitudinal cross-sectional view of an endotracheal tube in accordance with a second embodiment of the present invention looking at the tube in the same direction as in FIG. 1.

FIG. 4 is a longitudinal cross-sectional view of an endotracheal tube in accordance with a third embodiment of the present invention looking at the tube in the same direction as in FIG. 1.

FIG. 5 is a longitudinal cross-sectional view of an endotracheal tube in accordance with a fourth embodiment of the invention looking at the tube in the same direction as in FIG. 1.

FIG. 6 is a transverse cross-sectional view of the tube wall of FIG. 4 along line A-A.

FIG. 7 is a transverse cross-sectional view of the tube wall of FIG. 5 along line A-A.

FIG. 8 is a flow chart illustrating a method for locating the ETT of the present invention using ultrasound imaging.

### DETAILED DESCRIPTION OF THE PREFERRED EMBODIMENT(S)

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in other systems and methods. Before explaining the disclosed embodiments of the present invention in detail, it is to be understood that the invention is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Thus, for example, reference to “a contrast agent” may include a plurality of contrast agents and equivalents thereof known to those skilled in the art, and so forth. As well, the terms “a” (or “an”), “one or more” and “at least one” can be used interchangeably herein. It is also to be noted that the terms “comprising”, “including”, “composed of” and “having” can be used interchangeably.

For purposes of the present invention, the term “void,” as used herein, refers to voids or spaces of the same or different volumes located within a substrate, such as an endotracheal tube wall. These voids may be arbitrarily shaped, though in some embodiments, use of particular sizes may be desirable to further improve imaging.

As used herein, “contrast agent” refers to any medium, including liquids and gases that is echogenic, e.g. capable of enhancing the echo of a propagated ultrasound wave relative to an echo provided by tissue.

The present invention is directed to novel devices and methods for locating such devices in a patient using ultrasound imaging. Preferably, the method of location of the device can be carried out in real time. The devices of the present invention allow use of ultrasound imaging to identify, locate and monitor the devices in a patient using ultrasound imaging. This is accomplished by the incorporation of a plurality of voids in the device, which voids are suitable for visualization using ultrasound imaging.

One exemplary embodiment of the invention is an endotracheal tube (ETT) 10 and method for locating the endotracheal tube in a patient using ultrasound imaging, preferably in real time. The current invention enables the identification, location and ongoing monitoring of the location of an ETT 10 in the airway of a patient, especially a neonate, using ultrasound imaging. The incorporation of voids 4 in the tube wall 3 of the ETT 10 is employed to enhance the ultrasound image of ETT 10 to make it significantly easier to visualize. This improved ETT 10 allows a healthcare provider who is not an ultrasound imaging specialist to quickly and accurately confirm the location the ETT 10 in a patient's airway in real time.

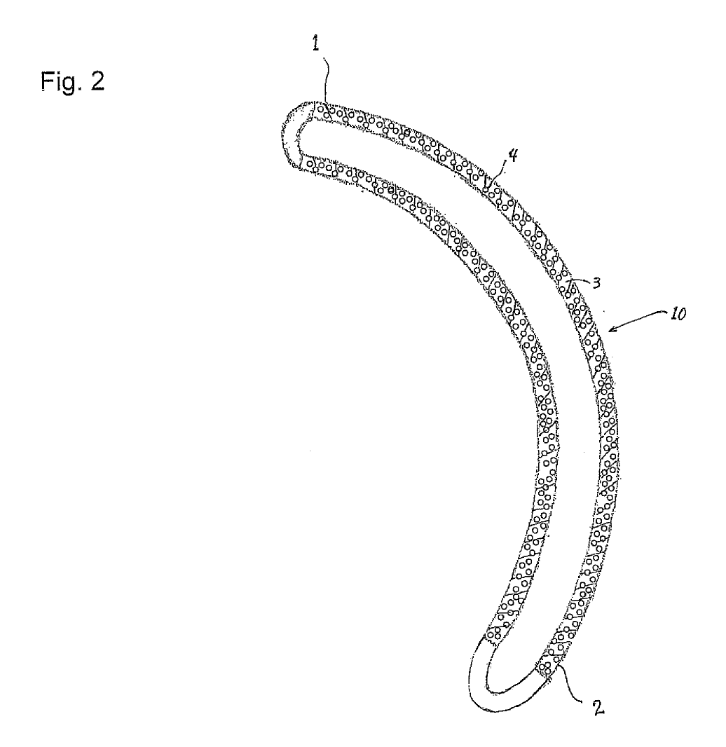

Referring to FIG. 2, a plurality of voids 4 located within tube wall 3 is provided in an amount which is sufficient to reflect ultrasound waves so that voids 4 are clearly viewable relative to the background in an ultrasound image. The reflection from the voids 4 as well as the reflection from the surfaces 6 and 7 of the ETT 10 make ETT 10 appear especially bright in an ultrasound image. The voids 4 also sharpen the contrast between ETT 10 and the tissue surrounding ETT 10. This is important because healthcare providers routinely use visual assessments of gray scale brightness to determine the location of an ETT 10 in an ultrasound image. The sharpened contrast between the image of ETT 10 and the surrounding tissue thus facilitates visualization of ETT 10 using this technique.

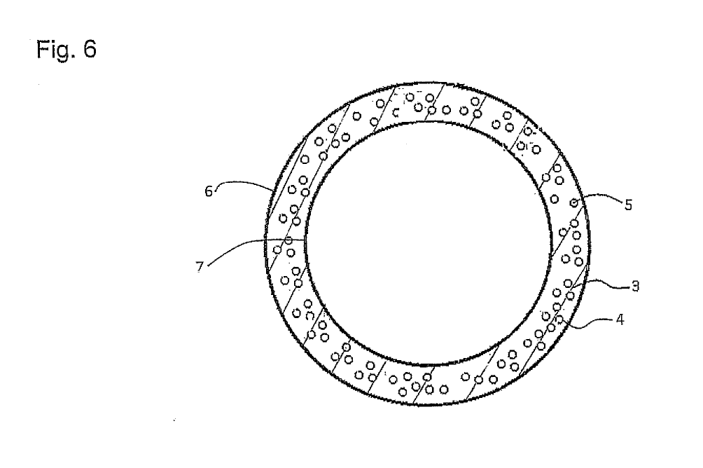

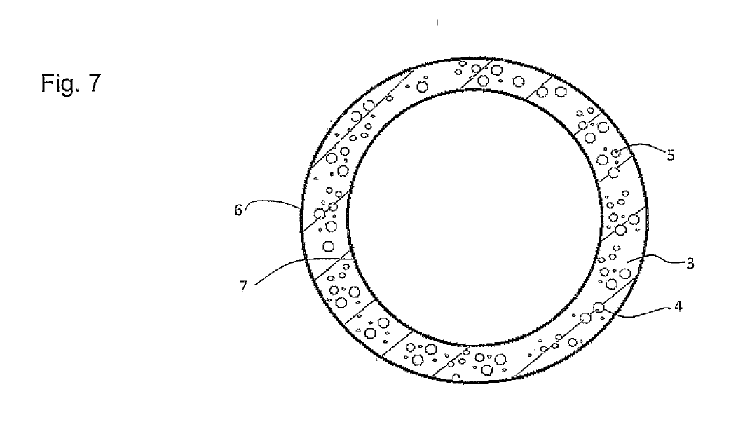

Voids 4 located in the tube wall 3 are preferably filled with ambient air since this is the natural result of a typical, inexpensive manufacturing process. However, it is possible to fill voids 4 with other materials, such as other liquids, gases or solids which may function as contrast agents 5 (shown in FIGS. 6-7)capable of enhancing the contrast between the space occupied by voids 4 and surrounding tissue in an ultrasound image. The ultrasound image quality of ETT 10 embedded with voids 4 may therefore be further improved relative to an ultrasound image of a conventional ETT through use of contrast agents, if desired. Any contrast agent capable of reflecting an ultrasound wave and enhancing the contrast between voids 4 and its surrounding environment is within the scope of this invention. Exemplary contrast agents may include air, inert gases, such as nitrogen, perfluorocarbons, such as perfluorobutane, perfluoropropane and perfluorohexane and combinations thereof Air is a useful contrast agent.

Voids 4 positioned within tube wall 3 may be arbitrarily shaped and have the same or different volumes. For example, the shape of voids 4 may be dictated by the manufacturing process used to fabricate ETT 10 with voids 4 therein for the purpose of minimizing the cost of manufacture of the device. In exemplary embodiments, the voids 4 may be spherical or elliptical. The volume of the voids may vary and can optionally be tailored for specific applications depending on the material used to fabricate ETT 10, as well as for the frequency of the ultrasound that will be used for imaging, if desired. The average diameter or largest dimension of the voids ranges from about 0.1 up to about 1000 micrometers, preferably from about 0.3 micrometer up to about 50 micrometers or 0.4 micrometer to about 10 micrometers.

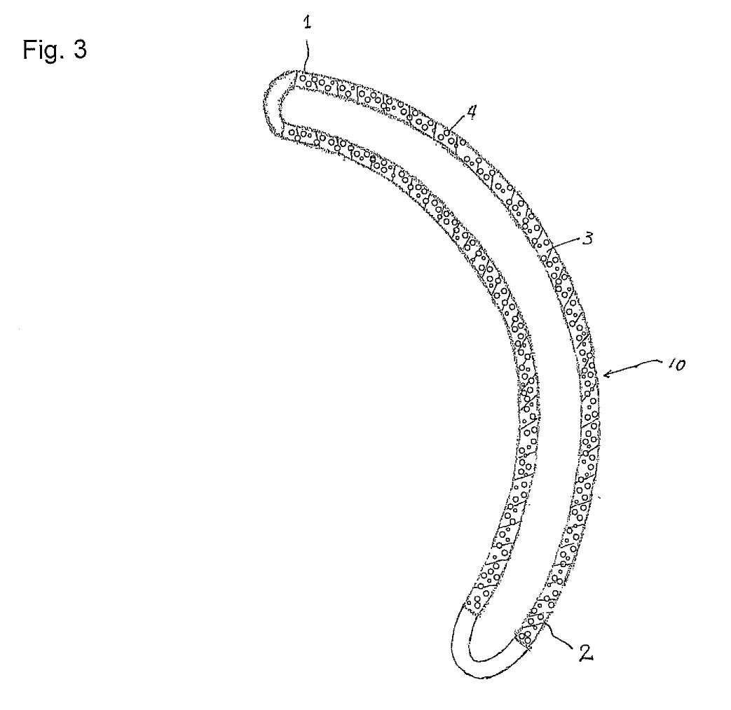

As shown in FIGS. 2-3, voids 4 are preferably configured as microspheres. The size of voids 4 may be correlated to the frequency of the propagated ultrasound wave in order to enhance ultrasound imaging of voids 4. Voids 4 of various sizes therefore may be incorporated in tube wall 3 of ETT 10 and the size of voids 4 may be selected to optimize echogenicity depending on the frequency of the ultrasound to be employed for imaging. This allows for customization of ETT 10 for particular ultrasound imaging devices by selection of a void size best suited for use with a particular ultrasound device.

Voids 4 may also be substantially uniform in size. As shown in FIGS. 2, 4, and 6, voids 4 have substantially the same shape and size, e.g. substantially the same diameter or largest dimension. In another embodiment, two or more voids 4 in tube wall 3 may have different sizes, as shown in FIGS. 3, 5 and 7, or three or more or a plurality of voids 4 located within tube wall 3 may have different sizes. In an exemplary embodiment, voids 4 are of mixed sizes between about 0.1 micrometer and about 1000 micrometers. ETTs 10 having voids of different sizes are capable of reflecting ultrasound waves over a wider frequency range thereby enabling their use with different frequencies of ultrasound, if needed. The advantage of this embodiment of ETT 10 is that the same ETT 10 can be used in conjunction with a variety of different ultrasound devices, even if the devices do not produce ultrasound at the same frequencies. Thus, ETTs 10 having varying sizes of voids 4 may be suitable for use in a larger set of circumstances and therefore reduce the cost of manufacturing and stocking different types of ETTs 10. This also eliminates the possibility of using an ETT 10 with voids of an unsuitable size for the frequency emitted by a particular ultrasound imaging device.

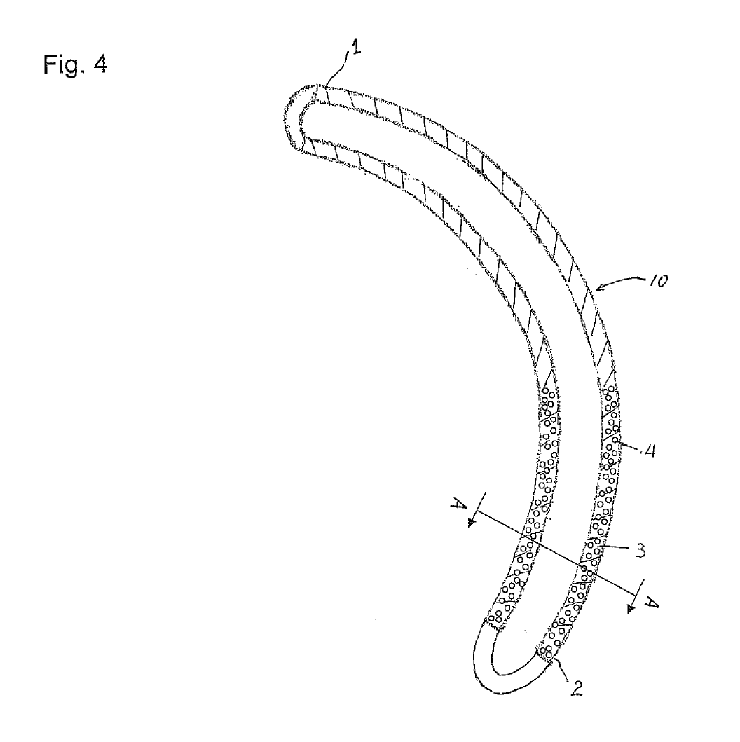

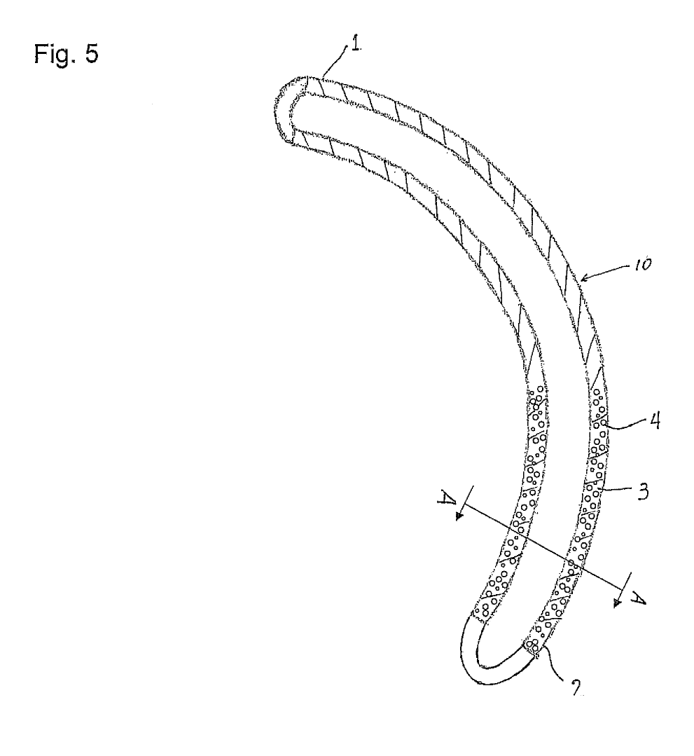

Referring to FIGS. 2 and 3, voids 4 may be uniformly or irregularly distributed within the tube wall 3 and arranged in any pattern and density to enable visualization of voids 4 in the airway of a patient using ultrasound imaging. In one embodiment, voids 4 are distributed throughout the length of tube wall 3, extending from the proximal end 1 to distal end 2 of the entire ETT 10. In another embodiment, voids 4 may be positioned within a portion of tube wall 3 that is proximate to the distal end 2, as shown in FIGS. 4-5.

This invention provides especially significant advantages for locating an ETT 10 in neonates as it does not require additional components that may cause trauma to the airway of a neonate or which may potentially separate from the body of the ETT 10 causing a choking hazard. In addition, neonates are very sensitive to slight shifts of the ETT 10 in the airway, and therefore require frequent confirmation of the ETT's 10 location. The current standard for determining the position of the ETT 10 in neonates, use of X-rays, requires multiple exposures of the neonates to ionizing radiation and thus is undesirable.

The invention may be appropriately sized and scaled for use in neonates, adults or larger children. For neonates, ETT 10 may have a length of about 10 cm and inner diameter ranging from about 2.5 millimeters to about 5 millimeters.

The present invention provides a number of advantages for identifying, locating and monitoring the position of ETT 10 within the airway of a patient. The device can minimize or eliminate the need for exposure to ionizing radiation by obviating the need to use X-rays to locate ETT 10. The device also improves the ability to visualize ETT 10 in an ultrasound image thereby allowing a larger universe of healthcare provides to perform ETT 10 location procedures since specialized training in ultrasound imaging should not be necessary to interpret the improved ultrasound images provided by the present invention. These same advantages can be realized for other devices that may be positioned in the body.

The present invention can be implemented in a variety of devices. Other exemplary devices which may include the same types of voids 4 and features described in relation to the ETT may include, catheter devices such as venous catheters, dialysis catheters, and percutaneously inserted central catheters, feeding tubes such as nasogatric tubes, devices for use in brachytherapy which need to be positioned for treatment, including expandable brachytherapy devices, and any other devices designed for insertion into the body where placement of the device is important for the use and/or functioning of the device.

In such devices, the voids 4 can be incorporated at any suitable location but are preferably incorporated in the device at key locations such as the tip of a catheter, the radiation delivery portion of a brachytherapy device, and the distal end of a feeding tube. The present invention is particularly suitable for devices that are placed in a body cavity or a blood vessel. For example, in a catheter, the voids 4 can be incorporated at one or more locations in the catheter tube or wall. Similarly, in a feeding tube, the voids 4 can be incorporated at one or more locations in the wall of the feeding tube. Other locations in such devices can also be employed as long as the user of the device can determine proper positioning of the device in the body from the information obtained by ultrasound imaging of the portion of the device which includes voids 4. In one embodiment, voids 4 are located proximate to a distal end of the device. In another embodiment, voids 4 are located along substantially the entire length of the device that is inserted into the body or patient to allow visualization of all or any portion of the device using ultrasound imaging when the device is located in a patient.

For brachytherapy devices, the voids 4 may be incorporated in any suitable portion of the brachytherapy device. For example, in devices which include tubes for delivery of radioactive materials to the treatment area, voids 4 can be incorporation at a location in one or more of such tubes which can be used for proper positioning of the brachytherapy device for treatment, e.g. at the beginning and/or end of the portion of the tubes which will be located in the treatment area. Alternatively, voids 4 can be located in a portion of the brachytherapy device which is at a fixed position relative to the treatment portion of the device. Though this embodiment is less preferred since it does not directly locate the treatment portion of the device in the body, indirect location can be employed using such devices. This may be desirable, for example, when the device includes materials that may interfere with the ultrasound imaging.

Another significant advantage of the present invention is that it allows the provision of a truly portable (e.g. mobile phone size, including battery) ultrasound device which can include a dedicated imaging transducer. Such a device is compatible for use in neonates, as well as older children or adults, as required. The device would have a small-footprint and minimal weight making it highly portable. This allows the device to be brought to the patient rather than requiring the patient to be brought to the device making use of the device easier and allowing the device to be used by paramedics or at remote locations, as needed.

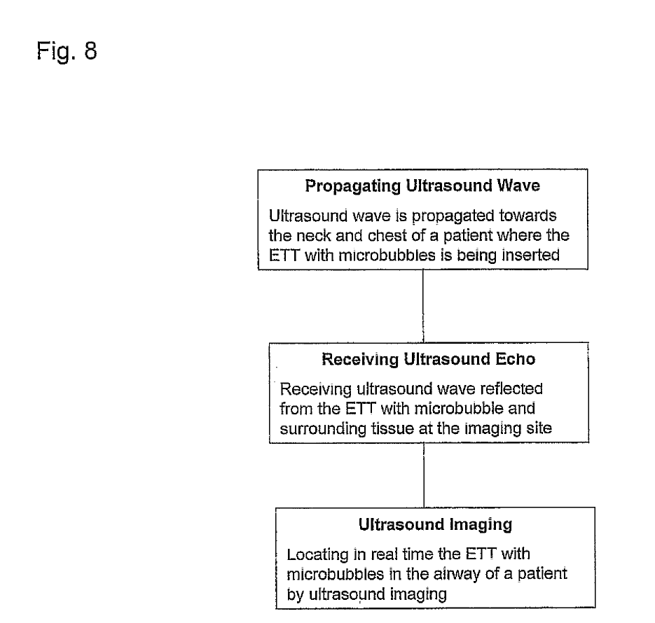

The present invention also relates to a method of using ultrasound imaging to detect the location of a device within a patient. This method is illustrated by a description of a method for location of an ETT 10 within an airway of a patient by visualizing voids 4 located within tube wall 3 of ETT 10. As described in FIG. 8, the method involves using an ultrasound device 8 to propagate ultrasound waves towards the neck or chest of a patient where ETT 10 is located. The propagated ultrasound waves are reflected by the gas, such as air, or other contrast agent contained in voids 4 of tube wall 3. These reflected ultrasound waves, e.g. ultrasound echoes, are received by ultrasound device 8 and an ultrasound imaging apparatus 9, which is preferably part of ultrasound device 8 or directly connected thereto, is used to generate an image of ETT 10 and at least some of the surrounding tissue and thereby allow a healthcare provider to locate ETT 10 within the patient. This method is also applicable to other devices for insertion in the body.

Ultrasound device 8 can be any ultrasound device, preferably, an ultrasound device of the latest generation. However, a portable ultrasound device is most preferred since this provides mobility for healthcare providers when they are trying to locate the device in a patient. This embodiment has the advantage of bring the ultrasound device to patients, instead of moving patients to the ultrasound device, because it may be undesirable, or even dangerous, to move the patient in some cases.

Wireless ultrasound devices 8 powered by batteries or another wireless power source, may be ideal for the present invention. Such wireless portable ultrasound devices 8, though they normally generate images of poor quality, are sufficient to visualize the device of the present invention due to its improved echogenicity. The present invention therefore offers great freedom in terms of mobility to healthcare providers and allows for real time imaging.

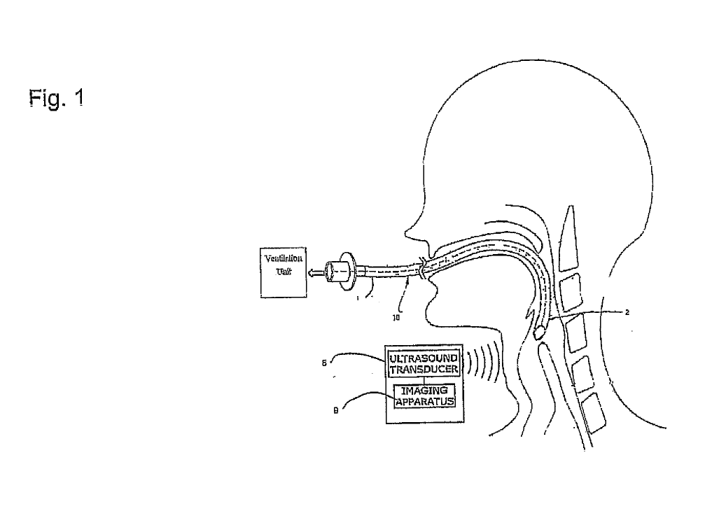

As discussed in above, the propagated ultrasound wave may have a frequency correlated to the size of the voids 4 to achieve enhanced echogenicity and improve visualization of the device. The method of present invention may call for an ultrasound device 8 that propagates ultrasound waves having a frequency best suitable for the size of the voids 4 in the device. For device having voids 4 of different sizes, as in FIGS. 2 and 4, the demand on ultrasound wave frequency is less, so ultrasound devices 8 with different wave frequency may be suitable for use. But for devices with voids 4 of approximately the same size, as in FIGS. 1 and 3, an ultrasound device 8 with a wave frequency most suitable for that particular void size is preferable. Therefore, for devices with uniformly sized voids 4, an ultrasound device 8 capable of emitting a correlated frequency is preferably selected. Alternatively, a device having voids of a size correlated to ultrasound device 10 may be selected.

Preferred propagated ultrasound wave frequencies range from about 1 MHz to about 40 MHz, corresponding to void sizes of from about 66 micrometers to about 0.16 micrometer in average diameter or largest dimension. In one embodiment, a mixture of frequencies ranging from about 1 MHz to about 40 MHz is employed in the propagated ultrasound wave. In an exemplary embodiment, voids 4 may be most effectively detected by using ultrasound imaging frequency determined from the following approximate expression: f[kHz]=6500/diameter of void in micrometers. When ultrasound devices 8 with wave frequencies outside of that preferred range are used, the void size would also be outside the preferred range, i.e. less than 0.16 micrometer or larger than 6.5 micrometers and thus, it is possible to use voids 4 of, for example, about 0.1-1000 micrometers in average diameter or largest dimension. The ultrasound imaging quality can be enhanced by an appropriate combination of ultrasound wave frequency, density and geometry of voids.

The present invention provides an improved method on locating an ETT 10 in the airway of a patient. First, the echogenicity of voids 4 in the device enable a real time, noninvasive, reliable way of visualizing the device in the patient. The sharper contrast between the device and surrounding tissue allows use of portable or wireless ultrasound devices for locating the device. This provides mobility to allow for use by paramedics or at the location of an accident or injury.

The present invention is especially useful in locating ETTs 10 in neonates. Because of the sensitivity of neonates, the small size of the airway in neonates and other safety concerns which significantly increase the need to locate and monitor the position of the ETT 10 in neonates, traditional methods of locating ETTs 10 in neonates are less than optimal for the reasons discussed above. With the enhanced contrast produced by the voids 4, ultrasound devices including portable or wireless ultrasound devices, can be used to reliably visualize ETTs 10 in neonates and obviate the need for invasive procedures or procedures relying on ionizing radiation.

It is to be understood that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

1. A device comprising:
a plurality of voids located in at least a portion of the device, wherein a number of said voids is sufficient for visualizing the device in a patient using ultrasound imaging.

2. The device of claim 1, wherein the device is sized for use in neonates.

3. The device of claim 1, wherein the voids are a plurality of different sizes.

4. The device of claim 1, wherein the voids are located in a portion of the device proximate to the distal end.

5. The device of claim 1, wherein the average diameter or largest dimension of said voids is up to about 1000 micrometers.

6. The device of claim 5, wherein the average diameter or largest dimension of said voids is from about 0.1 micrometer to about 1000 micrometers.

7. The device of claim 6, wherein the average diameter or largest dimension of the voids is from about 0.3 micrometer to about 50 micrometers.

8. The device of claim 6, wherein the average diameter or largest dimension of the voids is from about 0.4 micrometer to about 10 micrometers.

9. The device of claim 1, further comprising a contrast agent located in the voids, wherein said contrast agent is selected from the group consisting of air, nitrogen, perfluorobutane, perfluoropropane, perfluorohexane and combinations thereof.

10. The device of claim 1, wherein all of said voids are substantially the same size.

11. The device of claim 1, wherein said voids are located along substantially the length of the portion of the device that is inserted into a patient.

12. The device of claim 9, wherein said contrast agent is air.

13. The device of claim 1, wherein the device is selected from the group consisting of endotracheal tubes, catheters, feeding tubes and brachytherapy devices.

14. A method for locating a device in a patient comprising the steps of:
inserting into the patient a device having a plurality of voids located in at least a portion of the device,
propagating ultrasound towards the patient,
receiving an ultrasound echo from at least some of the voids and at least some surrounding tissue of the patient, and
creating an ultrasound image of the at least some of the voids and at least a portion of the surrounding tissue of the patient from said ultrasound echo, and
determining the location of the device in the patient from said ultrasound image.

15. The method of claim 14, wherein a portable ultrasound imaging device is used to propagate the ultrasound waves and receive the ultrasound echo.

16. (canceled)

17. The method in claim 14, wherein the propagated ultrasound waves comprise a mixture of frequencies in the range of about 1 MHz to about 40 MHz.

18. (canceled)

19. The method of claim 14, wherein all of the voids are substantially the same size.

20. The method of claim 14, wherein the voids are a plurality of different sizes.

21. The method of claim 14, wherein a size of the voids is selected based on a frequency of the propagated ultrasound wave.

22. The method of claim 14, wherein the device is selected from the group consisting of endotracheal tubes, catheters, feeding tubes and brachytherapy devices.
